(19) United States
(12) Patent Application Publication
(10) Pub. No.: US 2003/0083354 A1
(43) Pub. Date: May 1, 2003

(54) Phenylephrine tannate and pyrilamine tannate salts in pharmaceutical compositions

(75) Inventors: Jeffrey S. Kiel, Gainesville, GA (US); H. Greg Thomas, Villa Rica, GA (US); Narasimhan Mani, Gainesville, GA (US)

Correspondence Address: David E. Jefferies, Wood, Herron & Evans, L.L.P., 2700 Carew Tower, 441 Vine Street, Cincinnati, OH, 45202-2917, US

(73) Assignee: PediaMed Pharmaceuticals, Inc.

(21) Appl. No.: 10/047,578

(22) Filed: Oct. 26, 2001

(51) Int. Cl. A61K 31/44; A61K 31/7024; A61K 31/137

(52) U.S. Cl. 514/352; 514/649; 514/23

(57) ABSTRACT

Compositions of tannate salts of phenylephrine and pyrilamine produced by a method that allows for the in-situ conversion and incorporation of the tannate salts in a single dosage form. The conversion process includes dissolving a salts of phenylephrine and pyrilamine in a solvent and mixing with a dispersing agent and tannic acid to generate tannate salts. The tannate salts may be further processed to single dosage forms, such as tablets and suspensions.

### FIELD OF INVENTION

The present invention relates generally to the field of tannate chemistry and more specifically to methods for processing phenylephrine tannate and pyrilamine tannate compositions for use in the treatment of coryza and the compositions produced.

### BACKGROUND OF THE INVENTION

Pyrilamine and phenylephrine are well known, both in their free base form as well as salts, such as hydrochloride, citrate, maleate, tannate, etc. These compounds, when in the form of tannate salts, are particularly desirable due to their stability. As a result, they may be combined without any untoward side effects. The tannate salts have also been found to have better organoleptic properties such as taste, in comparison to other salts or free base forms of such compounds. In addition, tannate salts are relatively large molecules, which results in absorption over prolonged intervals of time. This reduces the frequency of administration of the compounds and thereby improves patient compliance factors. Due to the above properties, such compounds are amenable to use as active pharmaceutical ingredients in a composition.

Phenylephrine, known chemically as L-m-

hydroxy.alpha.[(methylamino)methyl] benzy alcohol, is a synthetic, optically active sympathomimetic compound, which has a 2°-amine functional group in its molecular structure. Phenylephrine hydrochloride is available as a white, odorless, non-hygroscopic, crystalline compound, in the form of the levorotayory isomer possessing a bitter taste. It is freely soluble in water and has a melting point of about 143° C.

Pyrilamine, one of the oldest and most enduring antihistaminic compounds, known chemically as N-[(4-methoxyphenyl)methyl]-N′, N′-dimethyl-N-2-pyridinyl-1,2-ethanediamine, and has a 3°-amine functional group present in its molecular structure and is an oily liquid. Pyrilamine hydrochloride is freely soluble in water, whereas the maleate salt is slightly soluble in water and has a melting point of about 101° C.

Tannic acid, also known as tannin, is a well-known naturally occurring substance typically produced from Turkish or Chinese nutgall. Chemically, these acids are described as polymers of different hydroxybenzoic acids. The chemistry of the tannins is complex and non-uniform. As a result the tannic acid used to produce antihistamine and decongestant tannate salts is variable in its purity. The water content of tannic acid varies form 5-10% and the molecular weight is about 1700.

Pyrilamine and phenylephrine in the form of their tannate salts are typically prepared by reacting the free bases of phenylephrine or pyrilamine, with tannic acid in the presence of a volatile solvent, usually isopropanol or water. The reaction mixture is stirred for about 1 hour while maintaining the mixture at 60-70° C. The reaction mixture is subsequently cooled to room temperature and then filtered, washed and vacuum dried to obtain the tannate salts. The yield of the tannate salt products using such methods typically varies from about 70% when using the isopropanol route to 90-97% using the water method. The purity of the tannate salts produced as described above is variable. The purity ranges form 85-90% when using the isopropanol route to about 90-98% when using the water route.

Due to the large nature of the tannate molecule, the percentage of antihistamine or decongestant free base within the tannate salt is significantly lower than that in other salt forms such as the hydrochloride or maleate. The presence of low active percentages of antihistamine or decongestant and the variable purity of the commercially available antihistamine and decongestant tannate salts results in the stoichiometry of the active free base to tannic acid in the tannate salts being different from batch to batch. This may result in significant dosing and processing problems during manufacture and increase the likelihood that commercially available pharmaceutical compositions contain variable, and in some instances, sub-therapeutic levels of active pharmaceutical ingredients.

Therefore, it would be desirable if pharmaceutical compositions containing pyrilamine and phenylephrine tannates could be prepared with reduced variability in active drug content and increased certainty that the active pharmaceutical ingredients are delivered within a therapeutic range.

### SUMMARY OF THE INVENTION

In accordance with the present invention and the contemplated problems which have and continue to exist in this field, the present invention provides a manufacturing method for in-situ conversion and incorporation of tannate salts of pyrilamine and phenylephrine in a single dosage form. The present invention also provides for pharmaceutical compositions including these tannate salts. These single dosage forms may include suspensions and tablets.

The present invention involves addition of a dispersing agent and tannic acid to purified water to which an aqueous solution of the active pharmaceutical ingredient, phenylephrine or pyrilamine, is added slowly to generate a water insoluble tannate salt. The presence of the dispersing agent prevents the clumping and aggregation of the tannate salt formed.

The resulting dispersion of the tannate salt in water may then be further processed by transferring to a suspending medium, whose composition includes thickening agents, sweetening/flavoring agents, anti-caking agents, co-solvents, pH adjusting agents, preservatives, coloring agents, and purified water. The resulting mixture can be processed into suitable liquid dosage forms, such as a suspension containing the tannate salts. In a preferred form, each 5 ml of the suspension contains 30 mg of pyrilamine tannate and 12.5 mg of phenylephrine tannate.

In an alternate method, pyrilamine and phenylephrine salts are dissolved in the water and a wet granulation is prepared by spraying the active ingredient solutions onto a mixture of tannic acid, dispersing agent and diluent. The granulation is subsequently dried and then is dry blended with additional diluent, and with sweetening, hardness-increasing, coloring, and flavoring agents as necessary. The resulting granulate can be processed into tablets. In a preferred form, the tablets contain 30 mg of pyrilamine tannate and 25 mg of phenylephrine tannate.

By starting with the commonly available salt or the free base form of the active pharmaceutical ingredient, which is subsequently converted and incorporated in-situ as a tannate salt, the invention provides an efficient, inexpensive, and reproducible method to manufacture products containing tannate salts as active ingredients.

By using the tannate salt of the active pharmaceutical ingredient, the present invention provides a dosage form which affords a sustained release of the active over prolonged intervals of time, and thereby improving patient compliance factors. Since the tannate salt of the active pharmaceutical ingredient is generated and incorporated in-situ into the dosage form during the manufacturing process, the purification and drying steps previously required for the isolation of the tannate salt are eliminated.

### DETAILED DESCRIPTION

In general, in a first embodiment, the invention features a manufacturing process for the in-situ conversion and incorporation of a combination of tannate salts of pyrilamine and phenylephrine into a therapeutic suspension dosage form which includes the steps of first dissolving the salts of the active pharmaceutical ingredients, pyrilamine and phenylephrine, at a temperature and pH value, that will not cause the composition to degrade. This forms a first salt solution. Pyrilamine and phenylephrine may be dissolved separately or together. Next, a dispersing agent is added to tannic acid in water, under stirring, to form a first dispersion. The next step involves transferring a part or whole of the first salt solution to the first dispersion under stirring to form a second solution including the tannate salts of pyrilamine and phenylephrine as a precipitate. Preferably, the salt solution is added to the first dispersion in small portions. Next, one adds thickening, suspending, coloring, sweetening, and flavoring agents to water under stirring, to form a third solution. One then combines preservative, pH adjusting, and anti-caking agents to glycerin under stirring to form a second dispersion. Adding the second dispersion in part or as a whole to the third solution under stirring, as a final mixing step, generates a suspension dosage form, preferably at a pH range of 3.5 to 6.5.

Pyrilamine and phenylephrine may be used as free bases or as salts having anionic functional groups of maleate, citrate, chloride, bromide, acetate, and sulfate. The source of the tannic acid used in the present invention may be natural or synthetic. The dispersing agent is chosen from the group consisting of magnesium aluminum silicate (MAS), xanthan gum and cellulose compounds.

In a particular embodiment, pyrilamine and phenylephrine are present in the composition in a range of about 0.05 to about 25.0% by weight. In yet another aspect, the step of dissolving the salts of the pyrilamine and phenylephrine in water at a maximum temperature that will not cause the composition to degrade is carried out in a temperature range of about 20° C. to about 50° C. In another aspect, the step of dissolving the salt of the pyrilamine or phenylephrine in water at a pH value, that will not cause the composition to degrade, is carried out at a pH in the range of 3 to 11. In yet another aspect, the dispersing agent is magnesium aluminum silicate (MAS) and is present in a range of about 0.05 to about 5.0% by weight, and the tannic acid is present in a range of about 0.05 to about 10.0% by weight. In another aspect of the present invention, the ratio of magnesium aluminum silicate to tannic acid by weight is in the range of about 0.1:1 to about 100:1. Additionally, in one aspect of the present invention, the ration of solid components to water by weight in the dispersion is in the range of about 1:25. Additionally, in one aspect, the ratio of tannic acid to the active pharmaceutical ingredients by weight is in the ratio of about 2:1 to about 10:1.

In another aspect of this embodiment of the present invention involving further processing of the tannate salts into a liquid dosage form, the addition of thickening, suspending, coloring, sweetening and flavoring agents to water under stirring to form a dispersion may occur where the thickening agent is magnesium aluminum silicate present in a range of about 0.5% to about 10.0% by weight; the suspending agent may be kaolin present in a range of about 0.5% to about 10.0% by weight; the sweetening agents may be sucrose and saccharin sodium present in a range of about 5.0% to about 50.0% and about 0.01 to about 3.0% by weight respectively; the flavoring agent is artificial grape and is present in a range of about 0.01 to about 1.0% by weight; and the dispersion medium is water and is present in a range of about 10.0% to about 85.0% by weight.

In another aspect of the method of the present invention, preservatives, pH adjusting, and anti-caking agents and glycerin may be combined under stirring to form the second dispersion, wherein the preservative used is methylparaben present in the range of about 0.01 to about 1% by weight; the pH adjusting agent is benzoic acid and is present in a range of about 0.05 to about 1% by weight; the anti-caking agent is pectin and is present in the range of about 0.5 to about 10% by weight; and the dispersion medium, glycerin, is present in the range of about 2.5 to about 20% by weight.

In the method of the present invention, the final pH of the suspension is in the range of 3.5 to 6.5. The final product is for immediate or sustained release of the active ingredients.

In one particular embodiment, the composition of the present invention is prepared for oral administration in the form of a liquid suspension formulated so that each 5 ml of suspension would contain 30 mg pyrilamine tannate and 12.5 mg phenylephrine tannate, when prepared by the methods of the present invention previously described. Table 1 below shows the initial starting ingredients and amounts for this particular embodiment of the invention.

TABLE 1

| Ingredient | % w/v | Wt. (mg/5 mL) |
|---|---|---|
| Pyrilamine Maleate | 0.32 | 16.00 |
| Phenylephrine HCl, USP | 0.10 | 5.00 |
| Tannic Acid, USP | 0.95 | 47.74 |
| Saccharin Sodium, USP | 0.05 | 2.53 |
| Sucrose, NF | 10.00 | 500.00 |
| Glycerin, USP | 7.53 | 376.47 |
| Magnesium Aluminum | | 80.00 |
| Silicate, NF | 1.60 | |
| Kaolin, USP | 1.61 | 80.59 |
| Pectin, USP | 1.50 | 75.00 |
| Methylparaben, NF | 0.20 | 10.00 |
| Benzoic Acid, USP | 0.10 | 5.00 |
| FD&C Red #40 | 0.02 | 0.76 |
| FD&C Blue No. 1 | 0.004 | 0.21 |
| Grape Flavor | 0.20 | 10.00 |
| Purified Water, USP | qs to volume | qs to volume |
| Total: | 100.00 | 850 L |

As noted in Table 1, the excipients used in the formulation are B sucrose, saccharin sodium and artificial grape flavor as flavoring agents; kaolin, pectin, magnesium aluminum silicate (MAS) as thickening and anti-caking agents; glycerin as a co-solvent; benzoic acid as a pH adjuster and a buffering agent; methylparaben as a preservative; FD&C Red No. 40 and FD&C Blue No. 1 as coloring agents; and purified water.

In this embodiment of the composition, the thickening agents kaolin and MAS, the flavoring agents sucrose, saccharin sodium and artificial grape and the coloring agents FD&C Red No. 40 and FD&C Blue No.1 are dispersed in purified water to generate the suspending medium. In particular, purified water is placed in a mixing tank and stirred. While stirring, the MAS is first added in small portions and mixed until a uniform dispersion is obtained. Similarly, the kaolin is transferred to dispersion. The saccharin sodium and the sucrose are then added and dissolved in the mixture. Mixing speed is adjusted to obtain a sufficient vortex to achieve the wetting of the MAS and kaolin and to minimize air entrapment. Typical mixing speeds may be between 500 and 1000 rpm.

The coloring agents FD&C Red No. 40 and FD&C Blue No. 1 are dissolved separately in water in a 600 ml beaker and added to the dispersion. The artificial grape flavor is then added to the mixture.

Mixing was continued until a uniform dispersion of all the ingredients was obtained. In the final formulation of this particular embodiment, pyrilamine tannate is present at 30 mg per 5 ml dose and phenylephrine tannate is present at 12.5 mg per 5 ml per dose.

As previously mentioned, the tannate salts of the active ingredients afford a more prolonged effect due to their slow dissolution. The most common and straightforward way of preparing the suspension is to use the tannate salt of the active as raw material. However, a different approach was adopted in one embodiment of the present invention. There, the free base of the active ingredient was converted in-situ into the tannate salt and then added to the suspension.

The active ingredients were obtained as hydrochloride and maleate salts. Phenylephrine was obtained as the hydrochloride and pyrilamine was obtained as the maleate salt.

The salts of the active ingredients were dissolved in purified water. This leads to the dissociation of the salt into its free base and conjugate acid forms. Another solution containing excess tannic acid in purified water was prepared. While stirring at low speeds, the solution of the salt was added in small portions to the tannic acid solution. Due to the presence of excess tannic acid, the free base reacts with the tannic acid to form the tannate salt. Since the tannate salt formed is larger in size and has low solubility in purified water, it is usually precipitated out of the solution.

In another embodiment, the present invention provides a manufacturing process for in-situ conversion and incorporation thereof, of pyrilamine and phenylephrine as tannate salts into suitable solid dosage forms such as tablets and capsules, for human and veterinary use. Since the tannate salt of the active is generated and incorporated in-situ into the dosage form during the manufacturing process, the isolation, purification and drying, routinely performed in the production of the commercially available tannate compounds, is eliminated.

In this embodiment, the present invention features mixing of a dispersing agent, a diluent and tannic acid, as dry powders, to generate a first powder mixture. An aqueous solution of salts of the active pharmaceutical ingredients (API), phenylephrine and pyrilamine is sprayed on or added slowly to the dispersing agent/tannic acid mixture to generate the tannate salt. The presence of the dispersing agent prevents the clumping and aggregation of the tannate salt formed and promotes uniformity in the first powder mixture. The tannate salt of the API obtained form the above conversion process, is then mixed with dry binding/matrix forming agents, and is wet granulated by spraying a solution of a binder. The granulation is subsequently dried, milled and then is dry blended with more diluent, sweetening, hardness increasing, coloring, flavoring and flow agents as necessary. The resulting granulate can be processed into tablets, capsules and other solid dosage forms as necessary.

The method of the present invention first involves the conversion of the API to the tannate salt by the reaction of functional groups in the molecular structure of the API, with tannic acid. The amount and ratio of dispersing agent and tannic acid is determined by the molecular configuration and concentration of the API. By starting with a commonly available salt of the API, which is subsequently converted and incorporated in-situ as a tannate salt, the invention provides an efficient method to manufacture solid dosage forms containing tannate salts as active ingredients.

Tannate pharmaceuticals referred to in this embodiment of the invention are solid dosage forms containing active pharmaceutical ingredients as tannate salts. These dosage forms are indicated for relief of nasal congestion and other allergies such as sinusitis, rhinitis and hay fever. The solid dosage forms include tablets (chewable and swallowable), capsules and the like. Owing to the large size of the tannate molecule, the absorption of the API is delayed and thereby the tablet provides a sustained effect due to the release of the active over prolonged intervals of time. By forming a tannate salt of the API, the present invention also improves taste, which improves patient compliance factors. In a particular embodiment, the present invention provides a method to produce a tablet formulation containing pyrilamine tannate and phenylephrine tannate as actives in a chewable tablet.

As with most pharmaceutical compositions, the compositions formed by the method of the present invention contain inert substances used as a diluent or vehicle for the drug. These excipients, in the present formulation, are as follows: avicel as a diluent, magnesium aluminum silicate (MAS) as a dispersing agent, corn starch as a binder, hydroxypropyl methyl cellulose (HPMC E-10) and xanthan gum as additional binding agents, calcium phosphate as a hardness enhancer, talc as a glidant, magnesium stearate as a lubricant, and grape flavor as a flavoring agent.

The first step of the method of the present invention is the conversion of the active pharmaceutical ingredients into tannate salts.

As previously mentioned the tannate salts of the active pharmaceutical ingredients afford a more prolonged effect due to their slow absorption. The simplest way of preparing the tablet is to use the tannate salt of the active pharmaceutical ingredients as raw material. However, the purity of the commercially available tannate compounds is variable. The stoichiometry of the free base to tannic acid in the raw material is different from batch to batch. This causes significant dosing and processing problems during manufacture.

Therefore, in the present manufacturing process, commonly available salts of the API, were converted in-situ into the tannate salt and subsequently incorporated into the tablet. Phenylephrine was obtained as a hydrochloride salt and pyrilamine was obtained as a maleate salt.

The salt forms of the active ingredients were dissolved in purified water. This resulted in the dissociation of the salt into its free base and conjugate acid forms. The tannic acid, MAS and avicel were blended as dry powders. While mixing the blend, the solutions of the active pharmaceutical ingredients were slowly poured onto the powders. A ten minute mixing time was allowed after addition of each active pharmaceutical ingredient. MAS present in the blend serves as a solid support to the tannic acid and aids in the dispersion of the tannate salt formed, thereby preventing any lumps that are formed as a result of the conversion process.

The granulate formed in the mixing process described above was dried at 45° C. and milled. The drying time was significantly reduced from that previously observed and there was a more uniform free flowing powder mass at the end of the drying step. The milled powder was then dry blended with additional avicel, calcium phosphate, talc, and magnesium stearate and was tableted. The granulate showed very good flow properties and a tablet hardness of 10-12 kp was obtained.

Based on the conversion step and properties such as flow, ease of blending, drying and milling of the granulation the concentration ranges of the excipients were as follows:

MAS: 0.10-4.50%

Calcium Phosphate: 1.00-3.00%

HPMC E-10: 1.00-3.00%

Avicel PH 102 (wet mass): 15.00-540.00%

Xanthan Gum: 1.50-7.50%

Corn starch: 0.50-2.00%

Talc: 0.30-1.00%

Magnesium stearate: 0.25-0.50%

In the case of the chewable tablets compressible sugar (Di-Pac) alternatively was used as a diluent to enhance the palatability of the tablet. The diluent was introduced in the dry blending stage of the formulation. The granulation manufactured using Di-Pac in the diluent showed good flow and tabletability. In addition, batches of the chewable tablets containing grape flavor were manufactured. However, those of skill in the art will recognize that any flavors may be used. The concentration ranges of the above excipients are as follows:

Di-Pac: 10.00-50.00%

Grape Flavor: 0.25-1.50%

The following Table 2 shows one embodiment of a formulation for composition made by the method of the present invention.

TABLE 2

| Ingredient | % w/w | Weight (mg) |
|---|---|---|
| Pyrilamine Maleate | 3.53% | 16.000 |
| Phenylephrine HCl | 2.22% | 10.000 |
| Tannic Acid, USP | 13.39% | 60.255 |
| Magnesium Aluminum | 3.00% | 13.500 |
| Silicate, NF | | |
| Avicel PH 102 | 39.99% | 179.955 |
| Sodium Saccharin | 1.00% | 4.500 |
| Methocel E-10M | 1.50% | 6.750 |
| Corn Starch | 1.00% | 4.500 |
| Di-Pac (Sucrose) | 28.72% | 129.240 |
| Calcium Phosphate | 2.25% | 10.125 |
| Dibasic | | |
| Xanthan Gum | 1.75% | 7.875 |
| Grape Flavor | 0.75% | 3.375 |
| Talc | 0.50% | 2.250 |
| Magnesium | 0.50% | 2.250 |
| Stearate | | |
| Total | 100.00% | 450.000 |

During the manufacture, a paddle blender was used which provided very good mixing of the powder during the conversion step and served to prevent the formation of the lumps in the formulation. The pyrilamine maleate and the phenylephrine hydrochloride solutions were poured onto the mixing powders and mixed for a period of 10 minutes to facilitate the conversion process. The granulation was dried at 45° C. and milled. The granulation was then dry blended with diluent, glidant, lubricant and the flavoring agent. The blend samples taken during the mixing showed good uniformity of the actives. The granulation exhibited good flow properties and medium oval tablets are 450 mg and 10-12 kp hardness were manufactured. Each tablet of this embodiment produced by the method of the invention includes 30 mg pyrilamine tannate and 25 mg phenylephrine tannate.

The principles of the present method of the invention will be more apparent with reference to the following Examples.

### EXAMPLE I

### Process of Conversion to Tannate Salts of Phenylephrine and Pyrilamine

The salt of the active ingredient, corresponding to an amount of free base present in a final batch size of 1 kg was dissolved in 100 ml of purified water. 120 ml of purified water was placed in a 600 ml beaker and stirred. While stirring, 3 g of MAS was added in small portions to obtain a dispersion. The amount of MAS used is a part of the total amount of MAS to be used in the formulation. Once the MAS was dispersed, tannic acid was added to the mixture and stirred to form a uniform dispersion. The amount of tannic acid used in the batch may vary from an amount equal to that of the free base, to three times that of the free base, present in the initial salt solution.

The salt solution was then added in small portions, under light stirring, to the MAS/Tannic acid dispersion. After all of the salt solution was added, the volume was made up to 250 ml with purified water and stirring was continued for a period of 10 minutes.

The MAS was used in this step to serve as an adherent or a solid support for the tannic acid molecules to facilitate the conversion process. In addition, it also prevented the clumping of the tannate salt formed, which aided in the dispersion of the precipitate of the tannate salt formed from the solution.

The pyrilamine salt solution, on addition to the MAS/tannic acid dispersion, resulted in the formation of copious amounts of precipitate. However, in the case of phenylephrine, the tannate salt showed partial solubility in purified water.

### EXAMPLE 2

### Process of Conversion to Tannate Salts of Phenylephrine and Pyrilamine

A solution of the active was prepared in the appropriate amount of purified water and a 2.0 g sample was removed for assay. The required amount of MAS was weighed and dispersed in purified water using a high shear mixer. Once the dispersion was uniform and lump-free, the tannic acid, at 3 times that of the free base of the active, was folded into the dispersion using a planetary mixer with a sweep blade. The sweeping action to disperse the tannic acid was found to significantly simplify the process, keep the tannic acid particles from clumping and provide greater uniformity of the dispersion. The salt solution was then added in small portions, while continuing to stir the MAS/tannic acid dispersion using the planetary mixer.

After all of the salt solution was added, the weight was made up to 800 g with purified water. Mixing was continued and 5 g samples of the conversion after 10, 20, and 30 minutes of mixing were taken in a centrifuge tube. The samples were subsequently centrifuged at 6500 rpm and the resulting supernatant solution was assayed for the presence of active.

At the end of 30 minutes, a 10 g sample of the conversion was taken for assay of the active.

The concentration of the excipients during the course of the above Experiments 1 and 2 were as follows: Sucrose was 10%, saccharin sodium and the flavoring agent were 0.05%. The total concentration of coloring agent is 0.02% (FD&C Red No. 40 was 0.019% and FD&C Blue No. 1 was 0.001%). Kaolin concentration was 1.6% and MAS concentration was 1.75%. Glycerin concentration was 7.5%. Methylparaben concentration was 0.2%. Benzoic acid concentration was 0.1%. Pectin concentration was 1.75%.

The pyrilamine maleate and the phenylephrine hydrochloride readily dissolved in water in the conversion step. From the weight obtained on a 20-gallon batch, the bulk density was found to be 1.01.

The foregoing is considered as illustrative only of the principles of the invention. Further, various modifications may be made of the invention without departing form the scope thereof and it is desired, therefore, that only such limitations shall be placed thereon as are imposed by the prior art and which are set forth in the appended claims.

What is claimed is:

1. A composition comprising the active pharmaceutical ingredients phenylephrine and pyrilamine, the composition formed from the steps of:
a. dissolving active pharmaceutical ingredients consisting of phenylephrine and pyrilamine in a first solvent to form a first solution, wherein dissolving said active pharmaceutical ingredients under conditions that will not cause decomposition of the active pharmaceutical ingredients;
b. mixing a dispersing agent and tannic acid in a second solvent to form a first dispersion;
c. transferring at least a portion of the first solution to the first dispersion, to form a second solution including tannate salts of the active pharmaceutical ingredients;
d. combining substances selected from the group consisting of preservatives, suspending agents, thickening agents, coloring agents, anti-caking agents, sweetening agents, flavoring agents and pH adjusting agents to form a liquid pharmaceutical carrier; and
e. combining at least a portion of the second solution to the liquid pharmaceutical carrier to produce a liquid dosage form including pharmaceutically active tannate salts.

2. The composition of claim 1 wherein the active pharmaceutical ingredients are present in a range of about 0.05% to about 25.0% by weight.

3. The composition of claim 1 wherein the active pharmaceutical ingredients are selected from the group of salts consisting of maleate, citrate, chloride, bromide, acetate, and sulfate.

4. The composition of claim 1 wherein the tannic acid is natural or synthetic.

5. The composition of claim 1 wherein the dispersing agent is selected from the group consisting of magnesium aluminum silicate, xanthan gum and cellulose compounds.

6. The composition of claim 5 wherein the dispersing agent is magnesium aluminum silicate and is present in a range of about 0.05% to about 5.0% by weight.

7. The composition of claim 1 wherein the tannic acid is present in a range of about 0.05 to about 10.0% by weight.

8. The composition of claim 6 wherein the magnesium aluminum silicate and tannic acid are present by weight in a ratio in the range of 0.1:1 to 100:1.

9. The composition of claim 1 wherein the tannic acid and the active pharmaceutical ingredients are present by weight in a ratio in the range of 2:1 to 10:1.

10. The composition of claim 1 wherein the thickening agent is magnesium aluminum silicate and is present in a range of about 0.5% to about 10.0% by weight.

11. The composition of claim 1 wherein the suspending agent is kaolin and is present in a range of about 0.5 to about 10.0% by weight.

12. The composition of claim 1 wherein the sweetening agents include sucrose present in a range of about 5.0% to about 50.0% by weight, and saccharin sodium present in a range of about 0.01% to about 3.0% by weight.

13. The composition of claim 1 wherein the flavoring agent is artificial grape and is present in a range of about 0.01% to about 2.0% by weight.

14. The composition of claim 1 wherein the second solvent is water and is present in a range of about 10.0 to about 75.0% by weight.

15. The composition of claim 1 wherein said second solvent is glycerin and is present in a range of about 2.5% to about 20.0% by weight.

16. The composition of claim 1 wherein the preservative is methylparaben and is present in a range of about 0.01 to about 1.0% by weight.

17. The composition of claim 1 wherein the pH adjusting agent is benzoic acid and is present in a range of about 0.05 to about 1.0% by weight.

18. The composition of claim 1 wherein the anti-caking agent is pectin and is present in the range of about 0.5 to about 10.0% by weight.

19. The composition of claim 1 wherein the pH of said liquid dosage form is in a range of about 3.5 to 6.5.

20. The composition of claim 1 wherein the pharmaceutically active tannate salts are pyrilamine tannate present at about 30 mg and phenylephrine tannate present at about 12.5 mg.

21. The composition of claim 19 wherein said liquid dosage form is a suspension.

22. A manufacturing process for the formation of a combination of pharmaceutically active tannate salts selected from the group consisting of phenylephrine and pyrilamine, which comprises the steps of:
a. dissolving active pharmaceutical ingredients consisting of phenylephrine and pyrilamine in a first solvent to form a first solution, wherein dissolving said active pharmaceutical ingredients occurs under conditions that will not cause decomposition of the active pharmaceutical ingredients;
b. mixing a dispersing agent and tannic acid in a second solvent to form a first dispersion;
c. transferring at least a portion of the first solution to the first dispersion, to form a second solution including tannate salts of the active pharmaceutical ingredients;
d. forming a liquid pharmaceutical carrier by combining substances selected from the group consisting of preservatives, suspending agents, thickening agents, coloring agents, anti-caking agents, sweetening agents, flavoring agents and pH adjusting agents; and
e. combining at least a portion of the second solution with the liquid pharmaceutical carrier to produce a liquid dosage form including pharmaceutically active tannate salts.

23. The process of claim 22, wherein forming a liquid pharmaceutical carrier further comprises combining suspending agents, thickening agents, coloring agents, sweetening agents, and flavoring agents in a third solvent to form a third solution.

24. The process of claim 23, wherein forming a liquid pharmaceutical carrier further comprises combining preservatives, anti-caking agents, and pH adjusting agents to a fourth solvent to form a second dispersion.

25. The process of claim 24, further comprising transferring at least a portion of the second solution to the third solution to form a third dispersion.

26. The process of claim 25, further comprising transferring at least a portion of the second dispersion to the third dispersion.

27. The process of claim 22, wherein the active pharmaceutical ingredients are provided as salts or in free base form.

28. The process of claim 22 wherein dissolving the active pharmaceutical ingredients in a first solvent occurs at a temperature in range of about 20 C to 50 C.

29. The process of claim 22 wherein dissolving an active pharmaceutical ingredients in a first solvent occurs at a pH in range of about 3 to 11.

30. The process of claim 22 wherein the liquid dosage form is for immediate or sustained release of the active ingredients.

31. A composition comprising active pharmaceutical ingredients selected from the group consisting of phenylephrine and pyrilamine, the composition formed from the steps of:
a. dissolving active pharmaceutical ingredients consisting of phenylephrine and pyrilamine in a first solvent to form a first solution, wherein dissolving said active pharmaceutical ingredient occurs under conditions that will not cause decomposition of the active pharmaceutical ingredients;
b. mixing a dispersing agent, diluent and tannic acid in a second solvent to form a first powder mixture;
c. transferring at least a portion of the first solution to the first powder mixture, to form tannate salts of the active pharmaceutical ingredients in a second powder mixture;
d. adding substances selected from the group consisting of dry binding/matrix forming agents and a binder solution to the second powder mixture in order to form a granulation;
e. combining the granulation with substances selected from the group consisting of diluent, coloring agents, sweetening agents, hardness-increasing agents, flavoring agents, and excipients; and
f. processing the granulation into solid dosage forms.

32. The process of claim 31 wherein the active pharmaceutical ingredients are free bases or salts selected form the group consisting of maleate, citrate, chloride, hydrochloride, bromide, hydrobromide, acetate, sulfate, mesylate, palmitate, and stearate.

33. The process of claim 31 wherein the tannic acid is natural or synthetic.

34. The process of claim 31 wherein the dispersing agent is selected from the group consisting of magnesium aluminum silicate, xanthan gum and cellulose compounds.

35. The process of claim 31 wherein the solvents are selected from the group consisting of purified water, ethanol, diethylether, methylene chloride, acetone, and isopropyl alcohol.

36. The process of claim 31 wherein the diluent is selected from the group consisting of lactose, microcrystalline cellulose, sucrose and mannitol and is present in a concentration of about 1.0 to about 75.0%.

37. The process of claim 31 wherein the binder solution comprises material selected from the group consisting of corn starch, pregelatinized starch, potato starch, polyvinylpyrrolidone and xanthan gum and is present in a concentration of about 0.1% to about 20.0%.

38. The process of claim 37 wherein the binder solution further comprises a solvent.

39. The process of claim 38 wherein the solvent is selected from the group consisting of purified water, ethanol, diethylether, methylene chloride, acetone, and isopropyl alcohol.

40. The process of claim 31 wherein the dry binding/matrix forming agents are selected from the group consisting of methylcellulose, hydroxypropyl methyl cellulose, ethylcellulose, hydroxypropyl cellulose, xanthan gum and polyvinyl pyrrolidone and each is present at a concentration of about 0.1% to about 20.0%.

41. The process of claim 31 wherein the coloring agents are selected from the group consisting of blue, red, yellow, green, orange, and purple and each is present at a concentration of about 0.01% to about 2.0%.

42. The process of claim 31 wherein the sweetening agents are selected from the group consisting of sucrose, saccharin sodium, xylitol and sucralose and each is present at a concentration of about 0.01% to about 40.0%.

43. The process of claim 31 wherein the flavoring agents are selected from grape, cherry, orange, lime and strawberry and is present in a concentration of about 0.01% to about 3.0%.

44. The process of claim 31 wherein the dispersing agent is magnesium aluminum silicate and is present in about 0.05% to about 15.0% by weight.

45. The process of claim 31 wherein the tannic acid is present in the range of about 0.05% to about 30.0% by weight.

46. The process of claim 44 wherein the ratio of magnesium aluminum silicate to tannic acid is present in the weight ratio of 0.1:1 to 100:1.

47. The process of claim 31 wherein the tannic acid and the active pharmaceutical ingredients are present in the weight ratio 2:1 to 10:1.

48. The process of claim 31 wherein the tannate salts are pyrilamine tannate present at 30 mg and phenylephrine tannate present at 25 mg.

49. A manufacturing process for the formation of a combination of pharmaceutically active tannate salts selected from the group consisting of phenylephrine and pyrilamine, as therapeutic solid dosage form for human use, which comprises the steps of:
a. dissolving active pharmaceutical ingredients consisting of phenylephrine and pyrilamine in a first solvent to form a first solution, wherein dissolving said active pharmaceutical ingredient occurs under conditions that will not cause decomposition of the active pharmaceutical ingredients;
b. mixing a dispersing agent, diluent and tannic acid in a second solvent to form a first powder mixture;
c. transferring at least a portion of the first solution to the first powder mixture, to form tannate salts of the active pharmaceutical ingredients in a second powder mixture;
d. adding substances selected from the group consisting of dry binding/matrix forming agents and a binder solution to the second powder mixture in order to form a granulation;
e. combining the granulation with substances selected from the group consisting of diluent, coloring agents, sweetening agents, hardness-increasing agents, flavoring agents, and excipients; and
f. processing the granulation into solid dosage forms.

50. The process of claim 49 wherein when combining excipients with the granulation the excipients are selected from the group consisting of calcium phosphate, calcium stearate, talc, colloidal silica, magnesium stearate and stearic acid and each is present at a concentration of about 0.1% to about 10.0%.

51. The process of claim 49 wherein dissolving the active pharmaceutical ingredient in a first solvent occurs at a temperature in the range of 30° C. to 50° C.

52. The process of claim 49 wherein dissolving the active pharmaceutical ingredient in a first solvent occurs at a pH in a range of 7 to 11.

53. A composition comprising tannate salts being formed by a method comprising:
a. dissolving active pharmaceutical ingredients selected from the group consisting of phenylephrine and pyrilamine in a first solvent to form a first solution, wherein dissolving said active pharmaceutical ingredients occurs at a temperature and pH value that will not cause decomposition of the active pharmaceutical ingredients;
b. mixing a dispersing agent and tannic acid in a second solvent to form a first dispersion; and
c. transferring at least a portion of the first solution to the first dispersion, to form a second solution including tannate salts of the active pharmaceutical ingredients.
